(19) United States
(12) Patent Application Publication
(10) Pub. No.: US 2018/0368765 A1
(43) Pub. Date: Dec. 27, 2018

(54) SYSTEMS AND METHODS FOR DETECTING AND MONITORING EXERCISE PRESSOR REFLEX IN HYPERTENSION

(71) Applicant: Boston Scientific Scimed, Inc., Maple Grove, MN (US)

(72) Inventors: Kyle H. Srivastava, St. Paul, MN (US); Craig M. Stolen, New Brighton, MN (US); Bryan A. Clark, Forest Lake, MN (US); Pramodsingh H. Thakur, Woodbury, MN (US)

(21) Appl. No.: 16/018,665

(22) Filed: Jun. 26, 2018

Related U.S. Application Data
(60) Provisional application No. 62/525,321, filed on Jun. 27, 2017.

(51) Int. Cl. A61B 5/00; A61B 5/021; A61B 5/11; A61B 5/0245; A61B 7/02; A61B 5/022; A61B 5/024; G16H 40/63

(52) U.S. Cl. CPC A61B 5/4884; A61B 5/021; A61B 5/1118; A61B 5/0245; A61B 7/02; A61B 5/0816; A61B 5/02416; A61B 5/0022; G16H 40/63; A61B 2562/0219; A61B 5/022

(57) ABSTRACT

The present disclosure relates generally to systems and methods for detecting and monitoring the exercise pressor reflex. In some embodiments, a medical system includes a first sensor connected to a user, wherein the first sensor detects an exertion metric of the user, and a second sensor connected to the user, wherein the second sensor detects a blood pressure metric of the user. The medical system may further include a processing component in communication with the first and second sensors, wherein the processing component is capable of processing the exertion metric and the blood pressure metric to determine an elevated blood pressure metric of the user based on a comparison between a baseline blood pressure metric and the detected blood pressure metric. The processing component may further determine an exercise pressor reflex (EPR) metric based on a comparison between the elevated blood pressure metric and the exertion metric.

### CROSS-REFERENCE TO RELATED APPLICATIONS

This application is a non-provisional application of, and claims the benefit of priority to, U.S. Provisional Application Ser. No. 62/525,321, filed Jun. 27, 2017, entitled “Systems and Methods for Detecting and Monitoring Exercise Pressor Reflex in Hypertension,” the entirety of which application is expressly incorporated by reference herein.

### FIELD

The present disclosure relates generally to the field of wearable and/or implantable medical devices and, more particularly, to wearable and/or implantable medical devices for detecting and monitoring exercise pressure reflex in hypertension.

### BACKGROUND

Hypertension is one of the strongest risk factors for developing cardiovascular disease. Chronic hypertension can lead to cardiac remodeling due to increased load on the heart and increases the patient's risk of developing heart failure. An exaggerated or elevated exercise pressor reflex (EPR) occurs when bouts of exercise raise the systolic blood pressure of a typically hypertensive patient, e.g., to over 200 mmHg. This happens when skeletal muscle afferents become overactive, resulting in an altered autonomic response to exercise, including increased blood pressure, heart rate, and systemic vascular resistance (SVR). Large spikes in blood pressure can result in cardiovascular, cerebrovascular, and/or organ damage. The exaggerated EPR differs from chronic hypertension, which is marked by baseline systolic blood pressure levels above 140 mmHg, and is particularly problematic for patients who would typically use exercise to improve blood pressure.

Blood pressure is often monitored at a baseline level in a clinician's office. However, because the EPR occurs during bouts of activity, which typically do not occur in a clinical setting, clinicians may be unaware of the extent of the problem in a patient. Furthermore, EPR is not often screened for in patients that do not have baseline hypertension, especially given current cardiopulmonary exercise (CPX) tests, which are high in costs and patient discomfort, thus leading to decreased use.

A variety of advantageous medical outcomes may therefore be realized by the device and/or methods of the present disclosure, which provide detection and monitoring of the EPR in hypertensive patients.

### SUMMARY

The present disclosure in its various embodiments relates generally to systems and methods for detecting and monitoring the exercise pressor reflex. In some embodiments, a medical system includes a first sensor connected to a user, wherein the first sensor detects an exertion metric of the user, and a second sensor connected to the user, wherein the second sensor detects a blood pressure metric of the user. The medical system may further include a processing component in communication with the first and second sensors, wherein the processing component is capable of processing the exertion metric and the blood pressure metric to determine an elevated blood pressure metric of the user based on a comparison between a baseline blood pressure metric and the detected blood pressure metric. The processing component may further determine an exercise pressor reflex (EPR) metric based on a comparison between the elevated blood pressure metric and the exertion metric.

In one or more embodiments, a medical system is disclosed that comprises a first sensor connected to a user, the first sensor detecting an exertion metric of the user; a second sensor connected to the user, the second sensor detecting a blood pressure metric of the user; and a processing component in communication with the first and second sensors. The processing component is capable of processing the exertion metric and the blood pressure metric to determine an elevated blood pressure metric of the user based on a comparison between a baseline blood pressure metric and the detected blood pressure metric, and to determine an EPR metric based on a comparison between the elevated blood pressure metric and the exertion metric. The processing component may be further capable of processing the exertion metric and the blood pressure metric to determine the EPR metric based on a comparison between an amount of change in the elevated blood pressure metric during a period of physical activity of the user, wherein the period of physical activity is determined based on the exertion metric. The EPR metric may be determined based on a comparison between the amount of change in the elevated blood pressure metric for a given amount of activity change identified during the period of physical activity. The amount of change in the elevated blood pressure metric during the period of physical activity of the user may be compared to a baseline amount of change in the elevated blood pressure. The processing component may be further capable of processing the exertion metric and the blood pressure metric to determine a rate of increase of the elevated blood pressure metric during the period of physical activity of the user. The processing component may be further capable of processing the exertion metric and the blood pressure metric to determine a rate of decrease of the elevated blood pressure metric during the period of physical activity of the user. The processing component may be further capable of processing the exertion metric and the blood pressure metric to determine a rate of decrease of the elevated blood pressure metric after the period of physical activity of the user has ended. The processing component may be further capable of processing the exertion metric and the blood pressure metric to determine a maximum elevated blood pressure metric for each of a plurality of distinct periods of physical activity of the user, and to compare the maximum elevated blood pressure metric to a baseline maximum elevated blood pressure metric. The processing component may be further capable of processing the exertion metric and the blood pressure metric to compare a change in the maximum elevated blood pressure metric to a change in the baseline maximum elevated blood pressure metric. The processing component may be further capable to compare the EPR metric to a predetermined baseline EPR metric, and to generate a notification when the EPR metric exceeds the predetermined baseline EPR metric. The processing component may be further capable to provide the notification to a storage device. The processing component may be further capable to provide the notification to at least one of the user, or a healthcare provider. The medical system may further comprise a wearable device. At least one of the first and second sensors may be integrated into the wearable device. The processing component may be provided within the wearable device. The processing component may be located remote from the wearable device. The first sensor may include at least one of an accelerometer, a gyroscope, or an electrocardiogram sensor. The second sensor may include at least one of a heart sound sensor, a sphygmomanometer, a photoplethysmogram device, or a physiological sensor.

In one or more embodiments, a medical system is disclosed that comprises a first sensor connected to a user, the first sensor detecting an exertion metric of the user; a second sensor connected to the user, the second sensor detecting a blood pressure metric of the user; and a processing component in communication with the first and second sensors. The processing component is capable of processing the exertion metric and the blood pressure metric to determine an elevated blood pressure metric of the user based on a comparison between a baseline blood pressure metric and the detected blood pressure metric, and to determine an EPR metric based on a comparison between an amount of change in the elevated blood pressure metric during a period of physical activity of the user, where the period of physical activity is determined based on the exertion metric. The first sensor may include at least one of an accelerometer, a gyroscope, or an electrocardiogram sensor. The second sensor may include at least one of a heart sound sensor, a sphygmomanometer, a photoplethysmogram device, or a physiological sensor.

In one or more embodiments, a method is disclosed that comprises detecting an exertion metric of a user by a first sensor; detecting a blood pressure metric of the user by a second sensor; receiving the exertion metric and the blood pressure metric at a processing component executing on a processor circuit; determining, by the processing component, an elevated blood pressure metric of the user based on a comparison between a baseline blood pressure metric and the detected blood pressure metric; and determining, by the processing component, an EPR metric based on a comparison between the elevated blood pressure metric and the exertion metric. The first sensor may include at least one of an accelerometer, a gyroscope, or an electrocardiogram sensor. The second sensor may include at least one of a heart sound sensor, a sphygmomanometer, a photoplethysmogram device, or a physiological sensor.

Various one or more of the features summarized above may be interchanged, exchanged, combined or substituted with or for other features summarized above, for use in connection with the medical systems and methods summarized above, and with respect to the embodiments described in greater detail below and embodiments otherwise within the scope of the present disclosure.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 is a diagram of a medical system according to embodiments of the present disclosure;

FIG. 2 is a block diagram of a medical system according to embodiments of the present disclosure;

FIG. 3 is a block diagram of a medical system according to embodiments of the present disclosure;

FIG. 4 depicts a graph of an exertion metric versus time according to embodiments of the present disclosure;

FIG. 5 depicts a graph of blood pressure metric versus time according to embodiments of the present disclosure;

FIG. 6 depicts a graph of a change in blood pressure metric versus a change in exertion metric according to embodiments of the present disclosure;

FIG. 7 is a flow chart of a method according to embodiments of the present disclosure;

FIG. 8 is a flow chart of a method according to embodiments of the present disclosure;

FIG. 9 is a block diagram of a storage device according to embodiments of the present disclosure; and

FIG. 10 is a block diagram of a computing architecture according to embodiments of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

The present disclosure relates generally to systems and methods for detecting and monitoring the exercise pressor reflex. In some embodiments, a medical system includes a first sensor connected to a user, wherein the first sensor detects an exertion metric of the user, and a second sensor connected to the user, wherein the second sensor detects a blood pressure metric of the user. The medical system may further include a processing component in communication with the first and second sensors, wherein the processing component is capable of processing the exertion metric and the blood pressure metric to determine an elevated blood pressure metric of the user based on a comparison between a baseline blood pressure metric and the detected blood pressure metric. The processing component may further determine an exercise pressor reflex (EPR) metric based on a comparison between the elevated blood pressure metric and the exertion metric.

Various embodiments herein may generally detect the periods and amounts of activity/exertion to quantify the EPR based on changes in blood pressure. The system can report one or more EPR metrics to the patient and/or a healthcare provider, or to another process (e.g. to a therapy device). Exemplary therapy devices may include neuromodulation therapy devices, such as spinal cord stimulation, vagus nerve stimulation, or sympathetic modulation therapy devices, a carotid baroreceptors stimulation device, a drug pump device, or other hypertension therapy devices, or combinations thereof. The system may be part of a wearable and/or implantable device configured to determine if the EPR is exaggerated in a patient.

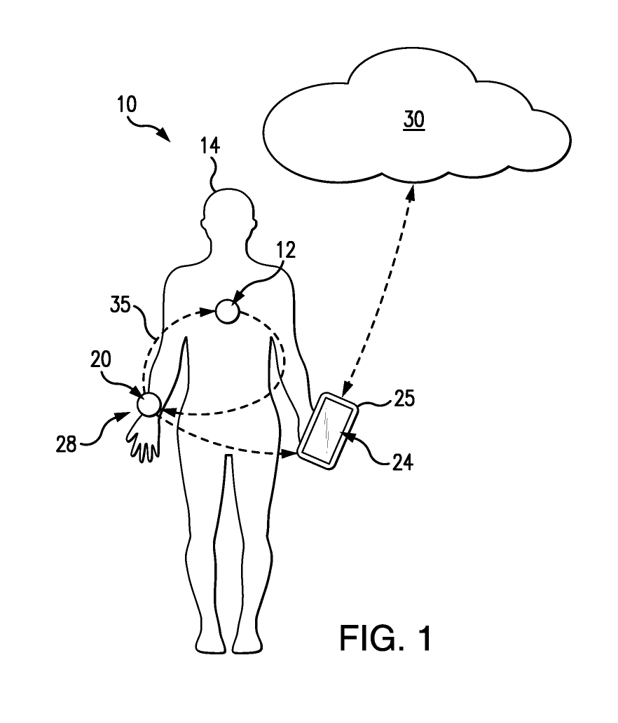

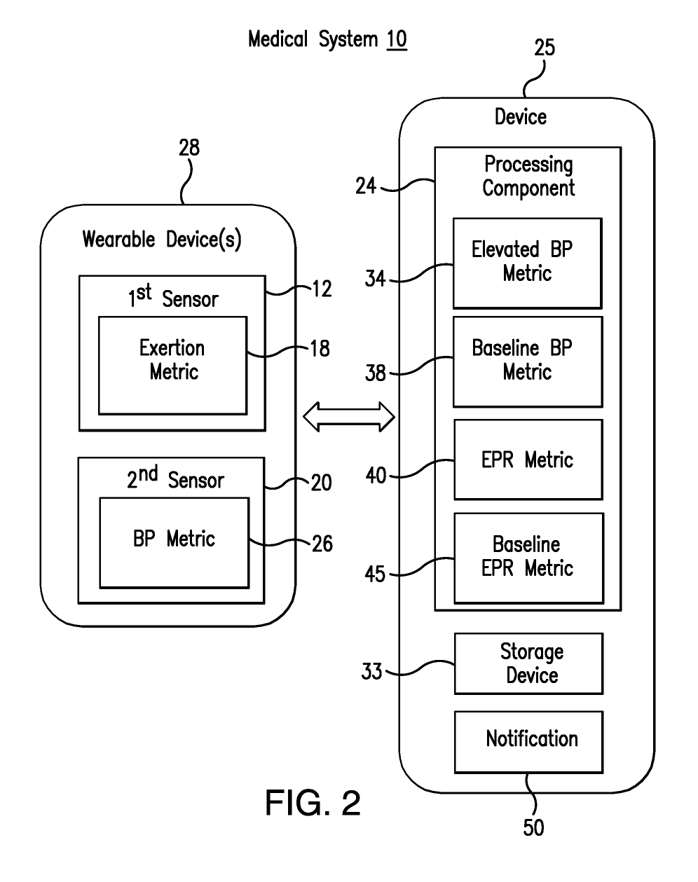

Turning now to FIGS. 1-2, a medical system 10 according to embodiments of the disclosure will be described in greater detail. As shown, the medical system (hereinafter “system”) 10 includes a first sensor 12 connected to a user 14, the first sensor 12 detecting an exertion metric 18 of the user 14. The first sensor 12 may include one or more functional/activity level sensors to measure activity level in the user 14, for example, by monitoring postural changes and movement. In one non-limiting embodiment, the first sensor 12 may include an accelerometer, which measures changes in position, wherein high levels of activity may be associated with rapid movements. The first sensor 12 may also include a gyroscope, which measures changes in roll, pitch, and yaw in order to determine angular acceleration. In yet other embodiments, the first sensor 12 may include a cardiovascular activity sensor, such as an electrocardiogram (ECG) sensor, which is capable of measuring heart rate as a surrogate for cardiovascular activity. An ECG sensor can also serve the purpose of a start time for measuring pulse wave velocity with a blood pressure sensor. In still yet other embodiments, the first sensor 12 may be a barometer/altimeter or a GPS. In still yet other embodiments, the first sensor 12 may include a respiratory sensor that may, for example, detect a respiratory rate or tidal volume, which may provide an indication of activity level of the user 14.

The system 10 further includes a second sensor 20 connected to the user 14, wherein the second sensor 20 detects a blood pressure (BP) metric 26 of the user 14. In one non-limiting embodiment, the second sensor 20 may include a heart sound monitor, which can be used to provide an indirect measure of blood pressure using sound differences detected in response to an aortic and/or pulmonic valve closure. In another embodiment, the second sensor 20 includes a sphygmomanometer (e.g., a blood pressure cuff), which directly measures blood pressure parameters. In yet another embodiment, the second sensor 20 may include a photoplethysmography (PPG) device for monitoring pulse transit time, pulse amplitude, normalized pulse volume, etc. The PPG may also be used to derive blood pressure measures such as systolic and diastolic pressure. In still yet other various embodiments, the second sensor 20 may include an electrical bioimpedance monitor, an impedance cardiograph monitor, or a physiological monitor, which measures additional physiological manifestations of peripheral perfusion or vascular resistance through invasive or noninvasive measures. Sensors that can measure physiological characteristics include flow sensors (e.g. ultrasonic flow sensor), perfusion sensors, and peripheral temperature sensors.

The system 10 may further include a processing component 24 in communication with the first sensor 12 and the second sensor 20. The processing component 24 may be a component within a device 25, such as a mobile device (e.g., computer and/or phone, tablet, etc.). In other embodiments, the processing component 24 may be part of a wearable device 28 containing the first sensor 12 and/or the second sensor 20. In yet other embodiments, the processing component 24 is located remote from the user 14, the wearable device 28, and/or the device 25. The processing component 24 communicates wirelessly with the first sensor 12, the second sensor 20, and a storage device 33, which may be located within the device 25 and/or a Cloud 30. In some embodiments, the storage device 33 provides longitudinal tracking of acquired signals to observe trends in physiological parameters and to record patient input, as well as EPR calculations. Cloud storage may be used to contain larger data sets for review by multiple parties, for example.

The processing component 24 is capable of receiving and processing the exertion metric 18 and the BP metric 26 to determine an elevated blood pressure metric 34 of the user 14 based on a comparison between a baseline BP metric 38, which may be retrieved from the storage device 33, and the detected BP metric 26. In some embodiments, the baseline BP metric 38 may be derived from a predefined clinical standard and/or an average BP metric obtained from a sample group of patients. In other embodiments, the baseline BP metric 38 is based on an average BP metric of the user 14, e.g., calculated from previous BP readings obtained over time.

The processing component 24 is further configured to determine an exercise pressor reflex (EPR) metric 40 based on a comparison between the elevated baseline BP metric 38 and the exertion metric 18. As will be described in further detail below, the processing component 24 is capable of processing the exertion metric 18 and the BP metric 26 to determine the EPR metric 40 based on a comparison between an amount of change in the elevated blood pressure metric 34 during a period of physical activity of the user 14. Based on a comparison of the EPR metric 40 to a predetermined baseline EPR metric 45, a notification 50 may be generated when the EPR metric 40 exceeds the predetermined baseline EPR metric 45, for example, by a specified percentage or numerical amount. The notification 50 may then be provided to user 14, e.g., through an alert generated on the device 25, and/or to a healthcare provider (not shown). In some embodiments, the predetermined baseline EPR metric 45 may be derived from a predefined clinical standard and/or an average EPR metric obtained from a sample group of patients. In other embodiments, the predetermined baseline EPR metric 45 may be based on previous readings obtained directly from the user 14.

In some embodiments, the processing component 24 may track and log, e.g., within the storage device 33, changes in the measured EPR metric 40 over time. For example, changes or trends in the EPR metric 40 could be recognized over the course of a day, a week, a month, etc. Based on additional patient information within the storage device 33, e.g., any therapy, medications, and/or medical conditions of the user 14, relationships may be patterned. This could be useful for determining a related therapy schedule (e.g. taking drug once or twice day), a therapy dose, therapy compliance, etc.

In some embodiments, the device 25 may permit input/control by the user 14, e.g., via a wireless communication system 35. For example, the device 25 may provide the user 14 an option to notify the processing component 24 to start/end exercise. Furthermore, the device 25 may communicate (e.g., visually and/or audibly) results of the EPR measurement, as well as report results of EPR measurement to a healthcare provider of the user 14.

In other embodiments, the device 25 may indicate to the user 14 an instruction or alert to perform a standardized exercise protocol, such as to climb a flight of stairs or walk twenty-five (25) meters, followed by a period of rest for a given number of minutes. The device 25 may include inputs for the user 14 to indicate that a standardized exercise protocol is being conducted, and may automatically determine periods for calculating the EPR. For example, if the patient takes a certain number of steps (e.g. 100) in some number of minutes (e.g. 3), the processing component 24 may measure how long it takes for the users' vitals (e.g. heart rate or blood pressure) to return to baseline values after activity has ended. If the time is too long or the change in the vital is too large, the device 25 logs that the EPR is exaggerated/unacceptable.

It will be appreciated that the device 25 may comprise any electronic device capable of receiving, processing, and sending information for the determination of the EPR. Examples of an electronic device may include without limitation a computer, a personal computer (PC), a desktop computer, a laptop computer, a notebook computer, a netbook computer, a handheld computer, a tablet computer, a server, a server array or server farm, a web server, a network server, an Internet server, a work station, a main frame computer, a supercomputer, a network appliance, a web appliance, a distributed computing system, multiprocessor systems, processor-based systems, wireless access point, base station, subscriber station, radio network controller, router, hub, gateway, bridge, switch, machine, or combination thereof. Embodiments herein are not limited in this context.

The device 25 may execute processing operations or logic for the determination of the EPR using the processing component 24. The processing component 24 may comprise various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

In some embodiments, the device 25 may execute communications operations or determination of the EPR using a communications component (not shown). The communications component may implement any well-known communications techniques and protocols, such as techniques suitable for use with packet-switched networks (e.g., public networks such as the Internet, private networks such as an enterprise intranet, and so forth), circuit-switched networks (e.g., the public switched telephone network), or a combination of packet-switched networks and circuit-switched networks (with suitable gateways and translators). The communications component may include various types of standard communication elements, such as one or more communications interfaces, network interfaces, network interface cards (NIC), radios, wireless transmitters/receivers (transceivers), wired and/or wireless communication media, physical connectors, and so forth. By way of example, and not limitation, communication media may include wired communications media and wireless communications media. Examples of wired communications media may include a wire, cable, metal leads, printed circuit boards (PCB), backplanes, switch fabrics, semiconductor material, twisted-pair wire, co-axial cable, fiber optics, a propagated signal, and so forth. Examples of wireless communications media may include acoustic, radio-frequency (RF) spectrum, infrared and other wireless media. The device 25 may communicate with other devices (not shown) via the Cloud 30.

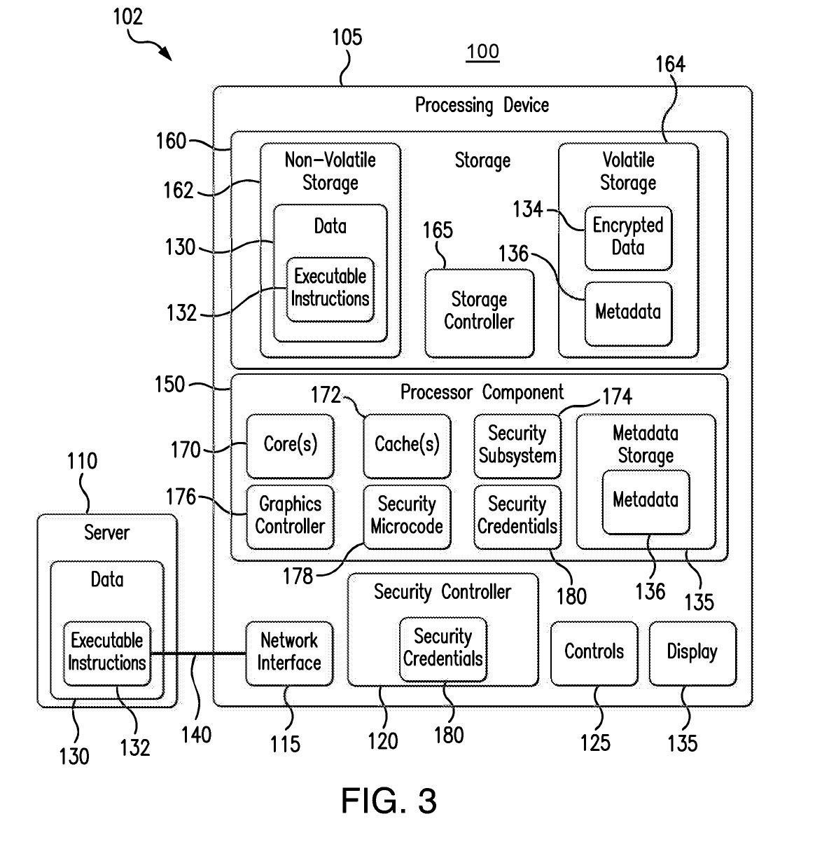

Turning now to FIG. 3, illustrated is an example of an operating environment 100 such as may be representative of some embodiments. In operating environment 100, which may be used to detect and monitor the exercise pressor reflex, a system 102 may include a server 110 and a processing device 105, which may be the same or similar to the device 25 of FIGS. 1-2, coupled via a network 140. Server 110 and processing device 105 may exchange data 130 via network 140, and data 130 may include executable instructions 132 for execution within processing device 105. In some embodiments, data 130 may be include data values, executable instructions, and/or a combination thereof. In other embodiments, data 130 may include sensor metric data from the first and second sensors 12, 20 of FIGS. 1-2. Network 140 may be based on any of a variety (or combination) of communications technologies by which signals may be exchanged, including without limitation, wired technologies employing electrically and/or optically conductive cabling, and wireless technologies employing infrared, radio frequency, and/or other forms of wireless transmission.

In various embodiments, processing device 105 may incorporate a processor component 150, which may be the same or similar to the processing component 24 of FIGS. 1-2, a storage 160, controls 125 (for instance, manually-operable controls), a display 135 and/or a network interface 115 to couple processing device 105 to network 140. Processor component 150 may incorporate security credentials 180, a security microcode 178, metadata storage 135 storing metadata 136, a security subsystem 174, one or more processor cores 170, one or more caches 172 and/or a graphics controller 176. Storage 160 may include volatile storage 164, non-volatile storage 162, and/or one or more storage controllers 165. Processing device 105 may include a controller 120 (for example, a security controller) that may include security credentials 180. Controller 120 may also include one or more of the embodiments described herein for unified hardware acceleration of hash functions.

Volatile storage 164 may include one or more storage devices that are volatile in as much as they require the continuous provision of electric power to retain information stored therein. Operation of the storage device(s) of volatile storage 164 may be controlled by storage controller 165, which may receive commands from processor component 150 and/or other components of processing device 105 to store and/or retrieve information therein, and may convert those commands between the bus protocols and/or timings by which they are received and other bus protocols and/or timings by which the storage device(s) of volatile storage 164 are coupled to the storage controller 165. By way of example, the one or more storage devices of volatile storage 164 may be made up of dynamic random access memory (DRAM) devices coupled to storage controller 165 via an interface, for instance, in which row and column addresses, along with byte enable signals, are employed to select storage locations, while the commands received by storage controller 165 may be conveyed thereto along one or more pairs of digital serial transmission lines.

Non-volatile storage 162 may be made up of one or more storage devices that are non-volatile inasmuch as they are able to retain information stored therein without the continuous provision of electric power. Operation of storage device(s) of non-volatile storage 162 may be controlled by storage controller 165 (for example, a different storage controller than used to operate volatile storage 164), which may receive commands from processor component 150 and/or other components of processing device 105 to store and/or retrieve information therein, and may convert those commands between the bus protocols and/or timings by which they are received and other bus protocols and/or timings by which the storage device(s) of non-volatile storage 162 are coupled to storage controller 165. By way of example, one or more storage devices of non-volatile storage 162 may be made up of ferromagnetic disk-based drives (hard drives) operably coupled to storage controller 165 via a digital serial interface, for instance, in which portions of the storage space within each such storage device are addressed by reference to tracks and sectors. In contrast, commands received by storage controller 165 may be conveyed thereto along one or more pairs of digital serial transmission lines conveying read and write commands in which those same portions of the storage space within each such storage device are addressed in an entirely different manner.

Processor component 150 may include at least one processor core 170 to execute instructions of an executable routine in at least one thread of execution. However, processor component 150 may incorporate more than one of processor cores 170 and/or may employ other processing architecture techniques to support multiple threads of execution by which the instructions of more than one executable routine may be executed in parallel. Cache(s) 172 may include a multilayer set of caches that may include separate first level (L1) caches for each processor core 170 and/or a larger second level (L2) cache for multiple ones of processor cores 170.

In some embodiments in which processing device 105 includes display 135 and/or graphics controller 176, one or more processor cores 170 may, as a result of executing the executable instructions of one or more routines, operate controls 125 and/or the display 135 to provide a user interface and/or to perform other graphics-related functions. Graphics controller 176 may include a graphics processor core (for instance, a graphics processing unit (GPU)) and/or component (not shown) to perform graphics-related operations, including and not limited to, decompressing and presenting a motion video, rendering a 2D image of one or more objects of a three-dimensional (3D) model, etc.

Non-volatile storage 162 may store data 130, including executable instructions 132. In the aforementioned exchanges of data 130 between processing device 105 and server 110, processing device 105 may maintain a copy of data 130, for instance, for longer term storage within non-volatile storage 162. Volatile storage 164 may store encrypted data 134 and/or metadata 136. Encrypted data 134 may be made up of at least a portion of data 130 stored within volatile storage 164 in encrypted and/or compressed form according to some embodiments described herein. Executable instructions 132 may make up one or more executable routines such as an operating system (OS), device drivers and/or one or more application routines to be executed by one or more processor cores 170 of processor component 150. Other portions of data 130 may include data values that are employed by one or more processor cores 170 as inputs to performing various tasks that one or more processor cores 170 are caused to perform by execution of executable instructions 132.

As part of performing executable instructions 132, one or more processor cores 170 may retrieve portions of executable instructions 132 and store those portions within volatile storage 164 in a more readily executable form in which addresses are derived, indirect references are resolved and/or links are more fully defined among those portions in the process often referred to as loading. As familiar to those skilled in the art, such loading may occur under the control of a loading routine and/or a page management routine of an OS that may be among executable instructions 132. As portions of data 130 (including portions of executable instructions 132) are so exchanged between non-volatile storage 162 and volatile storage 164, security subsystem 174 may convert those portions of data 130 between what may be their original uncompressed and unencrypted form as stored within non-volatile storage 162, and a form that is at least encrypted and that may be stored within volatile storage 164 as encrypted data 134 accompanied by metadata 136.

Security subsystem 174 may include hardware logic configured or otherwise controlled by security microcode 178 to implement the logic to perform such conversions during normal operation of processing device 105. Security microcode 178 may include indications of connections to be made between logic circuits within the security subsystem 174 to form such logic. Alternatively or additionally, security microcode 178 may include executable instructions that form such logic when so executed. Either security subsystem 174 may execute such instructions of the security microcode 178, or security subsystem 174 may be controlled by at least one processor core 170 that executes such instructions. Security subsystem 174 and/or at least one processor core 170 may be provided with access to security microcode 178 during initialization of the processing device 105, including initialization of the processor component 150. Further, security subsystem 174 may include one or more of the embodiments described herein for unified hardware acceleration of hash functions.

Security credentials 180 may include one or more values employed by security subsystem 174 as inputs to its performance of encryption of data 130 and/or of decryption of encrypted data 134 as part of performing conversions there between during normal operation of processing device 105. More specifically, security credentials 180 may include any of a variety of types of security credentials, including and not limited to public and/or private keys, seeds for generating random numbers, instructions to generate random numbers, certificates, signatures, ciphers, and/or the like. Security subsystem 174 may be provided with access to security credentials 180 during initialization of the processing device 105.

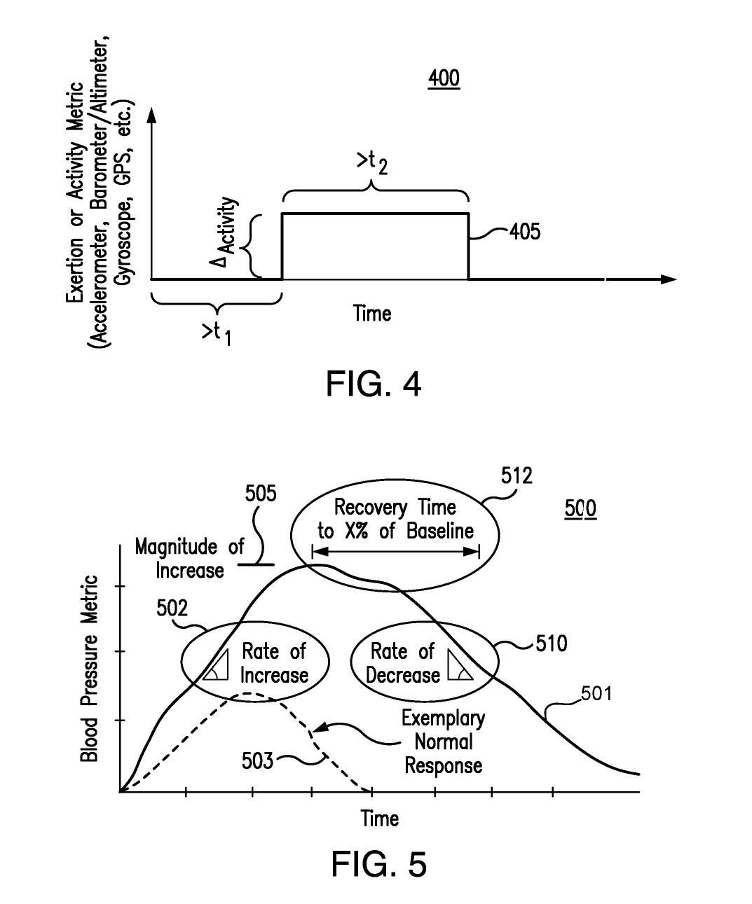

Turning now to FIGS. 4-6, various embodiments for determining EPR metrics and deviations from baseline EPR metrics will be described in greater detail. FIG. 4 depicts an example graph 400 of an exertion metric 405 versus time, wherein the exertion metric 405 may be the same or similar to the exertion metric 18 of FIG. 2. As shown, the exertion metric 405 is represented by a line linking a plurality of plotted data points over time, which may correspond to various periods of inactivity and activity of the user. The exertion metric 405 may be obtained by the first sensor 12, which may be an accelerometer, barometer/alternatoaltimeter, gyroscope, GPS, and the like. As shown at time t1, the first sensor 12 detects no exertion or activity. At the beginning of time t2, a change in physical activity of the user 14 is detected by the first sensor 12. At the end of time t2, the first sensor 12 again detects no exertion or activity. Although drawn as a step-change in activity level, it will be appreciated that the change may be more gradual/transient.

FIG. 5 depicts an example graph 500 of a BP metric 501 versus time, wherein the BP metric 501 may be the same or similar to the BP metric 26 of FIG. 2. As discussed above, the BP metric 501 may be obtained by the second sensor 20, which may be a heart sound sensor, a sphygmomanometer, a photoplethysmogram device, or a physiological sensor, and the like. As shown, the BP metric 501 is represented by a curve linking a plurality of plotted data points. Alternatively, the BP metric 501 represents the elevated blood pressure metric, for example, as compared to a normal/baseline BP metric 503. In this embodiment, as time increases, so does a rate of increase 502 of the curve (e.g., measured as slope) of the BP metric 501 until a maximum elevated blood pressure metric is achieved at point 505. After the maximum elevated blood pressure metric is achieved, the BP metric 501 decreases. As the BP metric becomes lower, a rate of decrease 510 of the curve (e.g., measured as slope) may be observed/calculated. In some embodiments, the recovery time to X % of a baseline BP metric may also be calculated, as shown at 512.

In example embodiments, the EPR metric may be determined based on a comparison between the amount of change in the elevated BP metric (e.g., 501) for a given amount of activity change (e.g., exertion metric 405) identified during the period of physical activity (e.g., t2). As discussed above, the EPR metric may be determined by a processing component, such as the processing device 25 of FIG. 2 and/or the processor component 150 of FIG. 3. In some embodiments, the processing component is further capable of processing the exertion metric 405 and the BP metric 501 to determine the amount of change in the elevated blood pressure metric during the period of physical activity of the user as compared to a baseline amount of change in the elevated blood pressure. For example, as shown in graph 500, the rate of increase and/or decrease 502/510 of the elevated blood pressure metric 501 during the period of physical activity of the user can be measured and compared to a rate of increase and/or decrease of the baseline BP metric 503 to determine if a deviation exists. In other embodiments, the processing component is further capable of processing the exertion metric 405 and the BP metric 501 to determine a rate of decrease of the elevated blood pressure after the maximum elevated blood pressure metric is achieved at point 505. In yet other embodiments, the processing component is further capable of processing the exertion metric 405 and the blood pressure metric 501 to determine a rate of decrease of the elevated blood pressure after the physical activity of the user has ended, for example, e.g., after time t2 shown in graph 400.

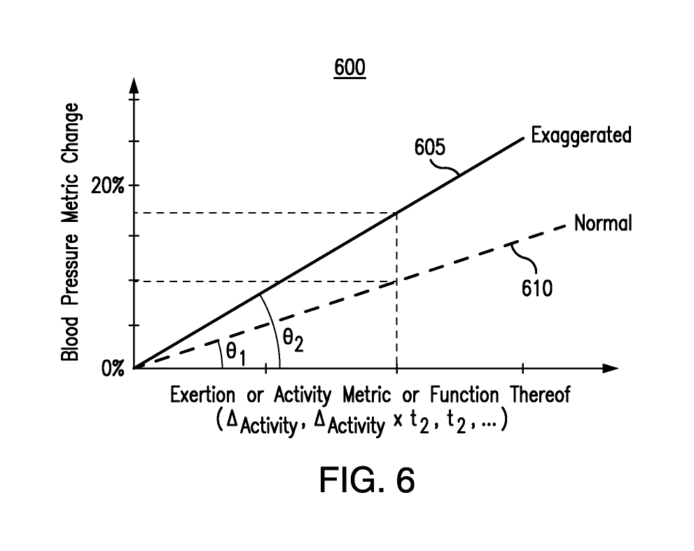

FIG. 6 depicts an example graph 600 of a BP metric change versus exertion metric. In this embodiment, a slope 605 of a max BP metric increase (e.g., as a percentage of the baseline BP metric) may be compared to quantified activity level increases over a plurality of distinct activity periods. More specifically, the processing component is capable of processing exertion metric and BP metrics to determine a maximum elevated blood pressure metric for each of a plurality of distinct periods of physical activity of the user. The processing component may then compare the maximum elevated blood pressure metrics to a normal/baseline maximum elevated blood pressure metric. A change in the maximum elevated BP metric, represented by slope 605, is compared to the change in the baseline maximum elevated BP metric, represented by slope 610. In some embodiments, the baseline maximum elevated BP metric is derived from a predefined clinical standard and/or an average obtained from a sample group of patients. A deviation between slope 605 and slope 610 may be quantified by a difference in angles between θ₁ and θ₂. In the event θ₁-θ₂ is greater than a predefined threshold, an unacceptable EPR may be present, and an alert or indication may be generated to alert the user and/or a medical professional.

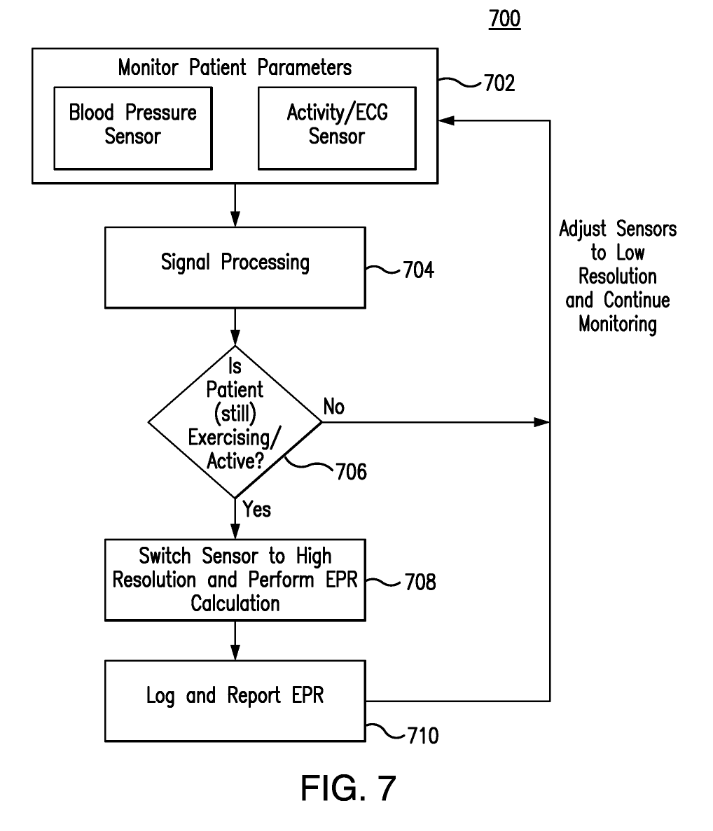

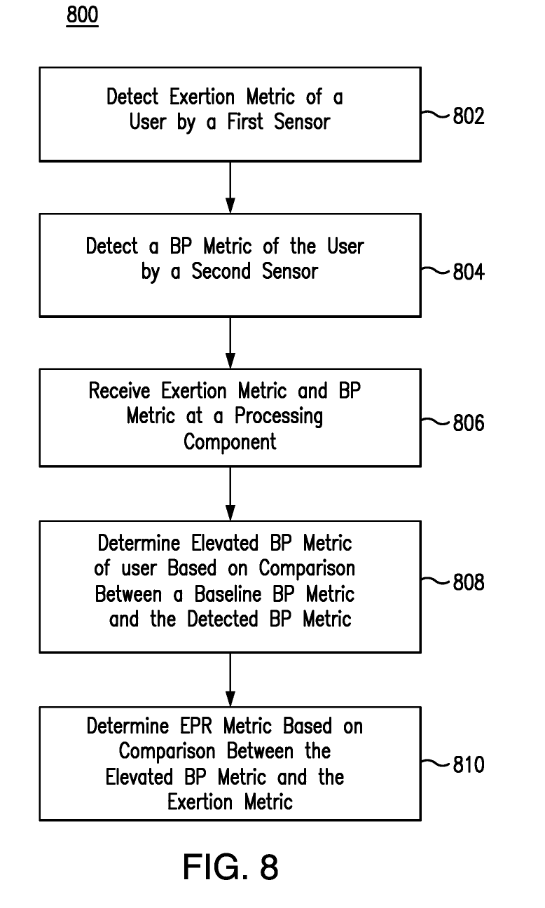

Turning now to FIGS. 7-8, one or more logic flows for carrying out methods of the disclosure may be provided. Although such figures presented herein may include a particular logic flow, it can be appreciated that the logic flow merely provides an example of how the general functionality as described herein can be implemented. Further, the given logic flow does not necessarily have to be executed in the order presented unless otherwise indicated. In addition, the given logic flow may be implemented by a hardware element, a software element executed by a processor, or any combination thereof. For example, a logic flow may be implemented by a processor component executing instructions stored on an article of manufacture, such as a storage medium. A storage medium may comprise any non-transitory computer-readable medium or machine-readable medium, such as an optical, magnetic or semiconductor storage. The storage medium may store various types of computer executable instructions, such as instructions to implement one or more disclosed logic flows. Examples of a computer readable or machine readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The embodiments are not limited in this context.

FIG. 7 depicts an illustrative logic flow 700 according to an embodiment. At 702, patient parameters are monitored. For example, a BP metric and an exertion metric may be determined based on data received from a BP sensor and an activity/ECG sensor, respectively. Signal processing of the BP metric and an exertion metric is performed at 704, and it is determined at 706 if the patient (user) is exercising and/or active. If not, the BP sensor and the activity/ECG sensor are adjusted to a lower resolution, and monitoring continues at 702. If the patient is exercising and/or active, the BP sensor and the activity/ECG sensor are adjusted to a higher resolution and the algorithm for calculating the EPR is performed at 708.

In some embodiments, the EPR is calculated according to the following approach. First, a baseline blood pressure level is calculated based on data from the BP sensor, e.g., either absolute blood pressure or a BP surrogate, such as a magnitude of a detected heart sound or a pulse wave velocity based on ECG and PPG/electrical bioimpedance/impedance cardiography. The baseline BP level may be obtained directly from the user, or may be a predetermined BP level obtained from a larger patient sampling and/or clinical standard. Second, periods of user activity and associated swings/changes in blood pressure or blood pressure surrogate are determined. In some embodiments, the periods of user activity include activity based on RMS levels from accelerometer and/or gyroscopes, activity based on mean heart rate from ECG or other blood pressure sensor, activity based at least in part on self-reported measures, e.g. via a smartphone application, or activity based on any combination of the above. Finally, at 710, the EPR is logged and reported (e.g., a notification is sent to the user and/or the user's doctor).

FIG. 8 depicts an illustrative logic flow 800 according to an embodiment. At 802, an exertion metric of a user may be detected by a first sensor. At 804, a BP metric of the user may be detected by a second sensor. At 806, the BP metric and the exertion metric may be received at a processing component executing on a processor circuit. At 808, the processing component may determine an elevated BP metric of the user based on a comparison between a baseline BP metric and the detected BP metric. At 810, the processing component may determine an EPR metric based on a comparison between the elevated BP metric and the exertion metric.

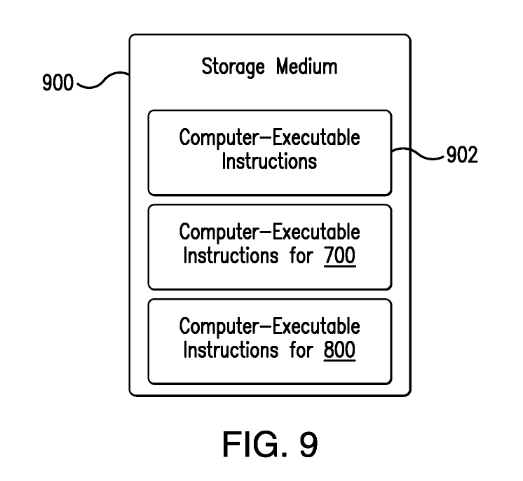

FIG. 9 illustrates an example of a storage medium 900. Storage medium 900 may comprise an article of manufacture. In some examples, storage medium 900 may include any non-transitory computer readable medium or machine readable medium, such as an optical, magnetic or semiconductor storage. Storage medium 900 may store various types of computer executable instructions, such as instructions 902, which may correspond to any embodiment described herein, or to implement logic flow 700 and/or logic flow 800. Examples of a computer readable or machine readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

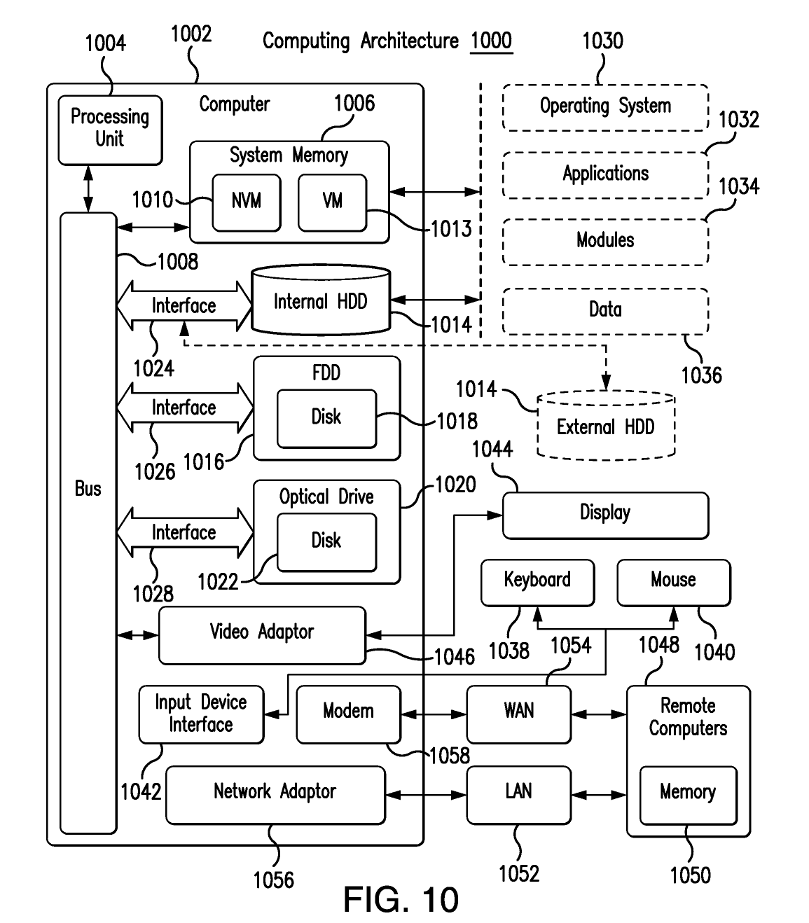

FIG. 10 illustrates an embodiment of an exemplary computing architecture 1000 suitable for implementing various embodiments as previously described. In one embodiment, the computing architecture 1000 may comprise or be implemented as part of an electronic device. Examples of an electronic device may include those described herein, such as device 25 of FIGS. 1-2 and processing device 105 of FIG. 3. The embodiments are not limited in this context.

As used in this application, the terms “system” and “component” are intended to refer to a computer-related entity, either hardware, a combination of hardware and software, software, or software in execution, examples of which are provided by the exemplary computing architecture 1000. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 1000 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 1000.

As shown in FIG. 10, the computing architecture 1000 comprises a processing unit 1004, a system memory 1006 and a system bus 1008. The processing unit 1004 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 1004. For example, the unified hardware acceleration for hash functions described herein may be performed by processing unit 1004 in some embodiments.

The system bus 1008 provides an interface for system components including, but not limited to, the system memory 1006 to the processing unit 1004. The system bus 1008 can be any of several types of bus structure that may further interconnect to a memory bus (with or without a memory controller), a peripheral bus, and a local bus using any of a variety of commercially available bus architectures. Interface adapters may connect to the system bus 1008 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computing architecture 1000 may comprise or implement various articles of manufacture. An article of manufacture may comprise a computer-readable storage medium to store logic. Examples of a computer-readable storage medium may include any tangible media capable of storing electronic data, including volatile memory or non-volatile memory, removable or non-removable memory, erasable or non-erasable memory, writeable or re-writeable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory 1006 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 10, the system memory 1006 can include non-volatile memory 1010 and/or volatile memory 1013. A basic input/output system (BIOS) can be stored in the non-volatile memory 1010.

The computer 1002 may include various types of computer-readable storage media in the form of one or more lower speed memory units, including an internal (or external) hard disk drive (HDD) 1014, a magnetic floppy disk drive (FDD) 1016 to read from or write to a removable magnetic disk 1018, and an optical disk drive 1020 to read from or write to a removable optical disk 1022 (e.g., a CD-ROM, DVD, or Blu-ray). The HDD 1014, magnetic FDD 1016 and optical disk drive 1020 can be connected to the system bus 1008 by a HDD interface 1024, an FDD interface 1026 and an optical drive interface 1028, respectively. The HDD interface 1024 for external drive implementations can include at least one or both of Universal Serial Bus (USB) and IEEE 1394 interface technologies.

The drives and associated computer-readable media provide volatile and/or nonvolatile storage of data, data structures, computer-executable instructions, and so forth. For example, a number of program modules can be stored in the drives and non-volatile memory 1010 and/or volatile memory 1013, including an operating system 1030, one or more application programs 1032, other program modules 1034, and program data 1036. In one embodiment, the one or more application programs 1032, other program modules 1034, and program data 1036 can include, for example, the various applications and/or components to implement the disclosed embodiments.

A user can enter commands and information into the computer 1002 through one or more wire/wireless input devices, for example, a keyboard 1038 and a pointing device, such as a mouse 1040. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 1004 through an input device interface 1042 that is coupled to the system bus 1008, but can be connected by other interfaces such as a parallel port, IEEE 1394 serial port, a game port, a USB port, an IR interface, and so forth.

A display 1044 is also connected to the system bus 1008 via an interface, such as a video adaptor 1046. The display 1044 may be internal or external to the computer 1002. In addition to the display 1044, a computer typically includes other peripheral output devices, such as speakers, printers, and so forth.

The computer 1002 may operate in a networked environment using logical connections via wire and/or wireless communications to one or more remote computers, such as a remote computer 1048. The remote computer 1048 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1050 is illustrated. The logical connections depicted include wire/wireless connectivity to a local area network (LAN) 1052 and/or larger networks, for example, a wide area network (WAN) 1054. Such LAN and WAN networking environments are commonplace in offices and companies, and facilitate enterprise-wide computer networks, such as intranets, all of which may connect to a global communications network, for example, the Internet.

When used in a LAN networking environment, the computer 1002 is connected to the LAN 1052 through a wire and/or wireless communication network interface or adaptor 1056. The adaptor 1056 can facilitate wire and/or wireless communications to the LAN 1052, which may also include a wireless access point disposed thereon for communicating with the wireless functionality of the adaptor 1056.

When used in a WAN networking environment, the computer 1002 can include a modem 1058, or is connected to a communications server on the WAN 1054, or has other means for establishing communications over the WAN 1054, such as by way of the Internet. The modem 1058, which can be internal or external and a wire and/or wireless device, connects to the system bus 1008 via the input device interface 1042. In a networked environment, program modules depicted relative to the computer 1002, or portions thereof, can be stored in a remote memory/storage device 1050. It will be appreciated that the network connections shown are exemplary and other means of establishing a communications link between the computers can be used.

The computer 1002 is operable to communicate with wire and wireless devices or entities using the IEEE 802 family of standards, such as wireless devices operatively disposed in wireless communication (e.g., IEEE 802.11 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

One or more aspects of at least one embodiment described herein may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A medical system, comprising:
a first sensor connected to a user, the first sensor detecting an exertion metric of the user;
a second sensor connected to the user, the second sensor detecting a blood pressure metric of the user; and
a processing component in communication with the first and second sensors, wherein the processing component is capable of processing the exertion metric and the blood pressure metric to:
determine an elevated blood pressure metric of the user based on a comparison between a baseline blood pressure metric and the detected blood pressure metric; and
determine an exercise pressor reflex (EPR) metric based on a comparison between the elevated blood pressure metric and the exertion metric.

2. The medical system of claim 1, the processing component further capable of processing the exertion metric and the blood pressure metric to determine the EPR metric based on a comparison between an amount of change in the elevated blood pressure metric during a period of physical activity of the user, wherein the period of physical activity is determined based on the exertion metric.

3. The medical system of claim 2, wherein the EPR metric is determined based on a comparison between the amount of change in the elevated blood pressure metric for a given amount of activity change identified during the period of physical activity.

4. The medical system of claim 2, wherein the amount of change in the elevated blood pressure metric during the period of physical activity of the user is compared to a baseline amount of change in the elevated blood pressure.

5. The medical system of claim 2, the processing component further capable of processing the exertion metric and the blood pressure metric to determine a rate of increase of the elevated blood pressure metric during the period of physical activity of the user.

6. The medical system of claim 2, the processing component further capable of processing the exertion metric and the blood pressure metric to determine a rate of decrease of the elevated blood pressure metric during the period of physical activity of the user.

7. The medical system of claim 2, the processing component further capable of processing the exertion metric and the blood pressure metric to determine a rate of decrease of the elevated blood pressure metric after the period of physical activity of the user has ended.

8. The medical system of claim 2, the processing component further capable of processing the exertion metric and the blood pressure metric to:
determine a maximum elevated blood pressure metric for each of a plurality of distinct periods of physical activity of the user; and
compare the maximum elevated blood pressure metric to a baseline maximum elevated blood pressure metric.

9. The medical system of claim 8, the processing component further capable of processing the exertion metric and the blood pressure metric to compare a change in the maximum elevated blood pressure metric to a change in the baseline maximum elevated blood pressure metric.

10. The medical system of claim 1, the processing component further capable to:
compare the EPR metric to a predetermined baseline EPR metric; and
generate a notification when the EPR metric exceeds the predetermined baseline EPR metric.

11. The medical system of claim 10, the processing component further capable to provide the notification to a storage device.

12. The medical system of claim 10, the processing component further capable to provide the notification to at least one of: the user, or a healthcare provider.

13. The medical system of claim 1, further comprising a wearable device, wherein at least one of the first and second sensors is integrated into the wearable device.

14. The medical system of claim 13, wherein the processing component is provided within the wearable device.

15. The medical system of claim 13, wherein the processing component is located remote from the wearable device.

16. The medical system of claim 1, wherein the first sensor includes at least one of an accelerometer, a gyroscope, or an electrocardiogram sensor; and the second sensor includes at least one of a heart sound sensor, a sphygmomanometer, a photoplethysmogram device, or a physiological sensor.

17. A medical system, comprising:
a first sensor connected to a user, the first sensor detecting an exertion metric of the user;
a second sensor connected to the user, the second sensor detecting a blood pressure metric of the user; and
a processing component in communication with the first and second sensors, wherein the processing component is capable of processing the exertion metric and the blood pressure metric to:
determine an elevated blood pressure metric of the user based on a comparison between a baseline blood pressure metric and the detected blood pressure metric; and
determine an exercise pressor reflex (EPR) metric based on a comparison between an amount of change in the elevated blood pressure metric during a period of physical activity of the user, wherein the period of physical activity is determined based on the exertion metric.

18. The medical system of claim 17, wherein the first sensor includes at least one of an accelerometer, a gyroscope, or an electrocardiogram sensor; and the second sensor includes at least one of a heart sound sensor, a sphygmomanometer, a photoplethysmogram device, or a physiological sensor.

19. A method comprising:
detecting an exertion metric of a user by a first sensor;
detecting a blood pressure metric of the user by a second sensor;
receiving the exertion metric and the blood pressure metric at a processing component executing on a processor circuit;
determining, by the processing component, an elevated blood pressure metric of the user based on a comparison between a baseline blood pressure metric and the detected blood pressure metric; and
determining, by the processing component, an exercise pressor reflex (EPR) metric based on a comparison between the elevated blood pressure metric and the exertion metric.

20. The method of claim 19, wherein the first sensor includes at least one of an accelerometer, a gyroscope, or an electrocardiogram sensor; and the second sensor includes at least one of a heart sound sensor, a sphygmomanometer, a photoplethysmogram device, or a physiological sensor.
